(19) United States
(12) Patent Application Publication
(10) Pub. No.: US 2026/0328727 A1
(43) Pub. Date: Oct. 22, 2026

(54) Detecting Defects in Metal Pipes

(71) Applicants: Saudi Arabian Oil Company, Dhahran (SA); King Abdullah University of Science and Technology, Thuwal (SA)

(72) Inventors: Ahmed Aljarro, Thuwal (SA); Moutazbellah Khater, Dhahran (SA); Tarek Mahmoud Atia Mostafa, Thuwal (SA); Guang An Ooi, Thuwal (SA); Mohammad Al-Ba’adani, Jeddah (SA); Mohmmed T. Abdulmohsin, Dhahran (SA); Fahad Otaibi, Alriyadh (SA); Khalid Ghamdi, Dhahran (SA); Salah A. Zahrani, Dammam (SA); Fahd Mohamed, Thuwal (SA); Hakan Bagci, Thuwal (SA); Shehab Ahmed, Thuwal (SA); Thamer Bahkly, Jeddah (SA)

(21) Appl. No.: 19/468,858

(22) Filed: Feb. 3, 2026

Related U.S. Application Data
(60) Provisional application No. 63/753,256, filed on Feb. 3, 2025.
(60) Provisional application No. 63/753,737, filed on Feb. 4, 2025.
(60) Provisional application No. 63/808,389, filed on May 19, 2025.

(51) Int. Cl. G01N 27/90; G01N 27/9013; G01N 27/904; G01N 29/04; G01N 29/22; G01N 33/2045

(52) U.S. Cl. CPC G01N 27/9006; G01N 27/902; G01N 27/904; G01N 29/04; G01N 29/225; G01N 33/2045; G01N 2291/0234; G01N 2291/0289; G01N 2291/2636

(57) ABSTRACT

A tool for pipe inspection includes: a low frequency eddy current (LFEC) sensor system mounted within the housing of the PIG around an outer circumference of the body of the PIG. The LFEC sensor system includes: a transmitter coil axially aligned with the body of the PIG; an electromagnetic source coupled to the transmitter coil; and a plurality of receivers positioned circumferentially around the transmitter coil with even spacing between each two adjacent receivers.

### CROSS-REFERENCE TO RELATED APPLICATIONS

This application claims the benefit of priority to U.S. Application No. 63/753,256, filed on Feb. 3, 2025; U.S. Application No. 63/753,737, filed on Feb. 4, 2025; and U.S. Application No. 63/808,389, filed on May 19, 2025, the contents of which are hereby incorporated by reference.

### TECHNICAL FIELD

This specification generally relates to detecting defects in metal pipes, particularly using eddy current systems.

### BACKGROUND

Pipes are important assets for transporting liquids or gases. Pipes can be exposed to harsh internal or external conditions that cause degradation in the quality of the pipes leading to corrosion, wall loss, or even leakage. Therefore, pipes are periodically inspected to assess the health of the pipe and to detect possible signs of damages before they become more severe. The inspection of pipes is usually carried out by a robot or a gadget that travels inside the pipes with the objective of detecting signs of defects while traveling through the pipe. These inspection practices are used in the oil and gas industry to assess the integrity of assets.

### SUMMARY

This specification describes an approach to detecting defects in metal pipes using eddy current systems transported on pipe inspection gauges (PIGs). Some systems include both high frequency eddy current (HFEC) and low frequency eddy current (LFEC) sensors. Some systems include both low frequency remote field eddy current (LF-RFEC) sensors and low frequency near field eddy current (LF-NFEC) sensors.

Systems combining HFEC and LFEC sensors can characterize inner diameter (ID) and outer diameter (OD) of metal pipes. The HFEC sensor interacts with and covers the inner ID of the metal pipe for the detection of inner and full wall losses. The LFEC penetrates the pipe thickness and detects external wall losses. The system is designed attachable to and/or integrable into off-the-shelf PIGs. Systems combining LF-RFEC sensors and LF-NFEC sensors can characterize the full range of external wall losses in terms of sensitivity and spatial resolution, i.e. sizes of surface area and depths of wall losses. Implementations of the described systems and methods can provide one or more of the following advantages.

The described systems and methods is able to detect both circumferentially and longitudinally extending defects. This capability can help provide comprehensive inspections in real-world scenarios where defects may occur in unpredictable patterns. The sensors' design and configurations are tuned to be agnostic to defects orientation, enabling detection of anomalies regardless of their alignment. This was also experimentally verified.

By combining near-field (NF) and remote-field (RF) configurations, this approach can provide improved accuracy in defect sizing and enhanced spatial resolution relative to system which only include one configuration or the other. The improved accuracy in defect sizing and enhanced spatial resolution is especially apparent for external wall losses.

These sensor configurations can provide full circumferential and axial coverage, minimizing detection gaps and maintaining high sensitivity. For example, LF-NFEC sensor systems have circumferentially distributed receivers with the transmitters are located at the center of the circle of receivers. In the LF-RFEC system, the transmitters and receivers are axially separated at fixed distances to keep the sensors are in the RFEC-dominant zone. This can improve electromagnetic (EM) field measurement to enhance defect characterization.

This approach does not require wire and shielding brushes to enhance eddy current sensing. This can reduce the complexity of the design of the tool and the nonlinearity of the detected EM fields to improve defect characterization performance.

The details of one or more embodiments of these systems and methods are set forth in the accompanying drawings and the description below. Other features, objects, and advantages of these systems and methods will be apparent from the description and drawings, and from the claims.

### DESCRIPTION OF DRAWINGS

FIGS. 1A and 1B are schematics of an eddy current system being used to monitor defects in a pipe.

FIG. 2 is a schematic view of the spatial relationship between receivers and a transmitter coil in a LFEC sensor system configured as a LF-NFEC sensor system.

FIGS. 3A and 3B are schematic views of the spatial relationship between the receivers and the transmitter coil in configurations of a LF-RFEC sensor system.

FIG. 4 is a schematic side view of a prototype LF-NFEC sensor system of the eddy current testing system.

FIG. 5 is a schematic view of the sensor configuration for a HFEC sensor system.

FIG. 6A is a perspective view of a prototype HFEC sensor system.

FIG. 6B is a more detailed view of a single HF sensor module.

FIG. 7 is a perspective view of the suspension wheels sensor system of the eddy current testing system of FIGS. 1A and 1B.

FIG. 8 is a schematic of a system with LF-RFEC sensors.

FIG. 9 is a schematic of a system with LF-RFEC and LF-NFEC sensors.

FIG. 10 is a schematic of a system with LF-RFEC, LF-NFEC, and HFEC sensors.

FIG. 11 is a schematic of a system with LF-RFEC, LF-NFEC, and HFEC sensors.

FIG. 12 is a block diagram illustrating an example computer system used to provide computational functionalities associated with described algorithms, methods, functions, processes, flows, and procedures according to some implementations of the present disclosure.

Like reference symbols in the various drawings indicate like elements.

### DETAILED DESCRIPTION

This specification describes an approach to detecting defects in metal pipes using eddy current systems mounted on PIGs. This approach based on uses both HFEC and LFEC sensors. By combining HFEC and LFEC sensors, this approach is able to characterize both the ID and the OD of metal pipes. The HFEC sensor interacts with and covers the inner ID of the metal pipe for the detection of inner and full wall losses. The LFEC penetrates the pipe thickness and detects external wall losses. The system is designed attachable to and/or integrable into off-the-shelf PIGs.

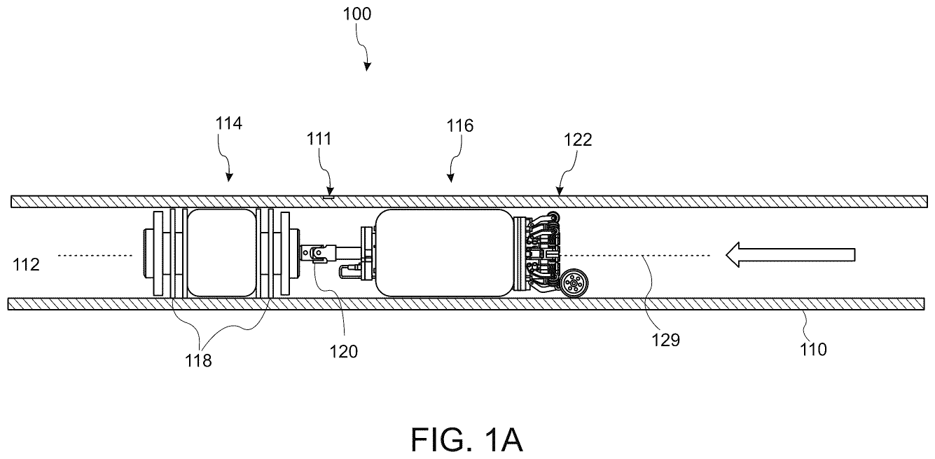

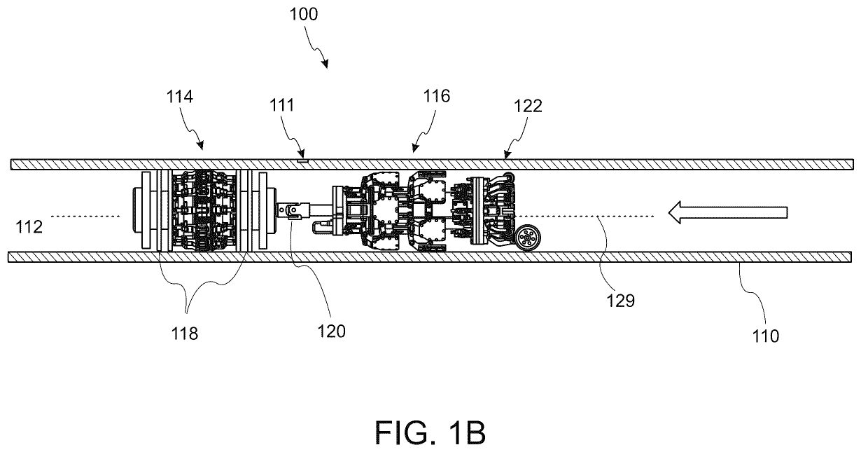

FIGS. 1A and 1B are schematics of an eddy current testing system 100 being used to monitor defects (e.g., defect 111) in a pipe 110 while being transported by a PIG 112. FIG. 1A shows the system 100 with its covers in place and FIG. 1B has the covers removed so internal components are visible. Flow in the pipe 110 is from right to left as indicated by the arrow in the pipe 110. The eddy current system 100 includes a LF-NFEC sensor system 114 and a HFEC sensor system 116 that interact with and characterize walls of the pipe 110. The LF-NFEC sensor system 114 is configured for penetration of the walls of the pipe 110 and can monitor the OD of the pipe for external wall losses based on electromagnetic field phases.

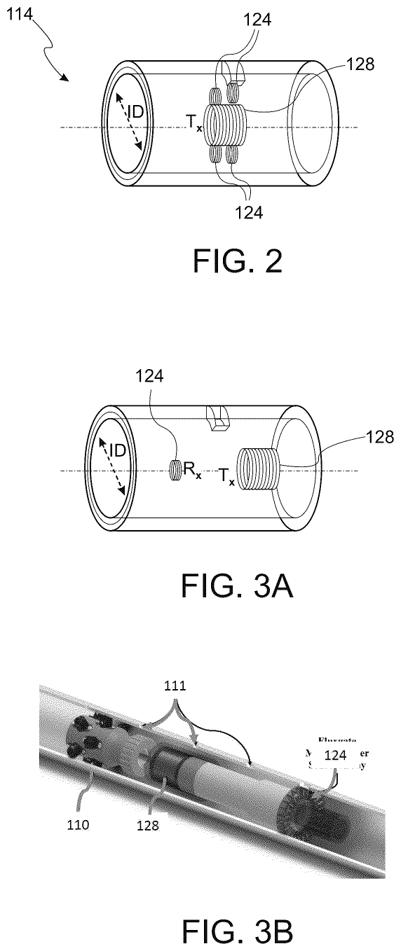

In eddy current testing system 100, the LFEC sensor system is a LF-NFEC sensor system 114. As explained with respect to FIGS. 2, 3A and 3B, the LFEC sensor system can be configured as a LF-NFEC sensor system or a LF-RFEC sensor system. Some LFEC sensor systems include both LF-NFEC and LF-RFEC configurations in a single sensor system. The HFEC sensor system 116 is configured to interact with and cover the ID of the pipe 110 for detection of inner and full wall losses based on electromagnetic field magnitude. Using only the dominant characteristics of the electromagnetic reduces complexity and improves performance. This refers to the complexity of the overall system for detecting defects, at the same time improving the sensitivity of the detection performance across the various and different types of defects.

The eddy current testing system is designed to be attached to and/or integrated into off-the-shelf PIGs. The PIG 112 is a standard cleaning PIG with sealing discs 118 on either end of the PIG. The LF-NFEC sensor system 114 is mounted around an outer circumference of the PIG 112 between the sealing discs 118 within a housing of the PIG. The HFEC sensor system 116 is attached to the PIG by a flexible joint 120 extending from a housing of the HFEC sensor system 116. The upstream end of the HFEC sensor system 116 is attached to a suspension wheels sensor system 122. This modular design allows operators to run each system independently. A prototype of the eddy current testing system 100 was produced and tested.

FIG. 2 is a schematic view of the spatial relationship between receivers 124 and a transmitter coil 128 in a LFEC sensor system configured as a LF-NFEC sensor system 114. In the illustrated sensor system, the receivers 124 are sensing coils. The transmitter coil 128 and the receivers 124 are located in a co-planar manner. The transmitter coil 128 is axially aligned with an axis 129 of the housing of the PIG and, consequently, with the center of pipelines the system is used in. Because they are aligned, the axis 129 is the axis of the body of the PIG, the axis of pipes in which the system is deployed, and the axis of the HFEC sensor system.

In contrast, receiver coils are distributed circumferentially with a pre-determined spacing between each two adjacent receiver coils at the same distance from the transmitter center. The receiver coils should be as close as possible to the pipe's inner surface for best detection performance. In some systems, the receivers 124 use other high sensitivity sensors rather than receiver coils.

This configuration allows for the possible detection of outer metal loss with a relatively compact inspection tool. It also allows LFEC sensor system 114 to operate effectively while allowing the device carrying the sensing sensor systems to travel at higher speed rates than systems using only an LF-RFEC sensor system. This is significant because the flow in some pipes can reach speeds up to 7 meters/second (m/s), requiring sensors to sample at 1 mm intervals. The eddy current testing system 100 uses advanced FPGA, or other ASIC, architectures to handle data flow efficiently and provide the extremely high-speed data acquisition and real-time processing capabilities needed to avoid data loss.

The transmitter coil 124 generates low-frequency electromagnetic fields, which induce eddy currents in the inspected pipes. These eddy currents produce a secondary magnetic field that can be measured by the receivers 128 and analyzed to characterize surface defects. The properties of the eddy currents that are induced in the pipe walls depend on the electromagnetic conductivity and permeability of the material, as well as the frequency and amplitude of the alternating field. The eddy currents experience a phase shift and attenuation as they flow through the pipe walls, the extent of which are determined by severity of wall defects. The behavior of electromagnetic fields is described by Maxwell's equations, which are a set of fundamental equations that relate the behavior of electric and magnetic fields to their sources. The relevant equations for low-frequency eddy current inspection are Faraday's law, Ampere's law, and Ohm's law, which can be combined to derive an expression for the eddy current density.

Faraday's law states that a changing magnetic field induces an electric field, which is given by:

$$∇ \times E = - \frac{\partial B}{\partial t}$$

where E is the electric field, B is the magnetic field, and t is time. Ampere's law states that the circulation of the magnetic field around a closed path is proportional to the current flowing through that path, which is given by:

$$∇ \times H = J + \frac{\partial D}{\partial t}$$

where H is the magnetic field intensity, J is the current density, and D is the electric displacement field. Ohm's law states that the current density is proportional to the electric field and the electrical conductivity, which is given by:

J=σE

where σ is the electrical conductivity. By combining these equations and assuming a sinusoidal time dependence, it is possible to derive an expression for the eddy current density:

J(x,ω)=jωσ(x)E(x,ω)

where j is the imaginary unit, ω is the angular frequency, σ(x) is the electrical conductivity as a function of position, and E(x,ω) is the electric field as a function of position and frequency. This expression relates the eddy current density to the electrical conductivity and the electric field, both of which can be measured and analyzed to detect surface defects in the material. The eddy current density can also be used to calculate the secondary magnetic field produced by the eddy currents, which can be measured and analyzed to detect surface defects.

In practice, the secondary magnetic field produced by the eddy currents is detected using a magnetic sensor, such as a coil of wire or a fluxgate magnetometer. Any anomalies in the measured values as the sensor moves along the surface of the pipeline are used to detect and characterize defects. The presence of surface defects such as wall losses, corrosion, or other types of damage affects the magnetic field produced by the eddy currents. These defects alter the electrical conductivity and magnetic permeability of the material, causing changes in the eddy current density and the secondary magnetic field. These changes can be detected and measured using a magnetic sensor, which is moved along the surface of the pipeline. The magnetic sensor measures the secondary magnetic field at various locations, and the measured values are plotted as a function of the distance traveled along the pipeline. This creates a magnetic field profile that can be analyzed to detect and characterize any surface defects. Surface defects in the pipeline produce anomalies in the magnetic field profile, which can be used to identify the position, location, shape, and extent of the defect. The amplitude and shape of the anomaly depend on the type and severity of the defect, as well as the operating conditions of the inspection system.

The transmitter coil 128 is driven by a LF source, typically on the order of hundreds of hertz (Hz). The low operating frequency allows for the detection of outer metal loss, but the sensitivity of receiver coils may be compromised at very low frequencies. This method requires employing very sensitive receivers or precision magnetic sensors in order to detect and distinguish the target outer metal loss.

Experimental tests were carried out to assess different frequencies and sensors. Three sensing elements tested included solenoid coils with a ferrite core, GMR sensors AA002 from NVE Corporation, and fluxgate magnetometers DRV425 from Texas Instruments. The solenoid coils with ferrite core are electromagnetic coils that use a ferromagnetic core to increase their magnetic field strength. The GMR sensors are solid-state devices that use the giant magnetoresistance effect to measure changes in magnetic field strength. The fluxgate magnetometers are devices that use a closed loop of ferromagnetic core with an excitation coil and two pick-up coils to measure the magnetic field.

Fluxgate, and scalar magnetometers are not typically used in NDT since they are larger, cost more, and have limited measuring range and bandwidth. However, the frequency of operation of the described RFEC system is low and the transmitter-receiver spacing is long. This reduces the need for sensors with extensive range and large bandwidth increasing the viability of using fluxgate magnetometers, especially modern fluxgate magnetometers with reduced sizes and weights. Using an array of miniature single-axis fluxgate magnetometers as a receiver enhances measurement sensitivity and spatial resolution.

Each of the sensing elements was tested oriented in both axial and radial directions with respect to the pipe's inner surface. In the axial direction, the sensing vector was parallel to the pipe's inner surface, while in the radial direction, the sensing vector was perpendicular to the pipe's inner wall. This allowed investigation of the effects of the orientation on the sensitivity and accuracy of each sensing element.

By operating at the higher end of the LF-NFEC frequencies, the device carrying the sensing sensor systems can travel at higher speeds, which can accommodate for the native field conditions with high product flow rate. This approach allows LF-NFEC systems to detect of outer metal loss via compact designs and tool sizes. However, the higher proximity of the receivers 124 to the transmitter coil 128 in LF-NFEC systems can lead to less sensitivity in the detection of the smaller sized anomaly features when operating at the higher end of the LF range (i.e., 2000 Hz). The systems described in this specification address this challenge by employing very sensitive receivers and precision magnetic sensors, in addition to appropriate data processing techniques to reliably detect and distinguish these outer pipe anomalies.

FIGS. 3A and 3B are schematic views of the spatial relationship between the receivers 124 and the transmitter coil 128 in two configurations of a LF-RFEC sensor system. In the illustrated sensor system, the receivers 124 are sensing coils. The transmitter coil 128 and the receivers 124 are axially aligned at the center of the pipeline. The system illustrated in FIG. 3A has a single receiver 124 while the system illustrated in FIG. 3B has an array of smaller receivers 124.

The transmitter coil 128 is driven by a lower frequency source, typically in the order of 1-100 Hz. The receiver(s) 124 are located at an axial distance from the transmitter coil 128 equal to 1.25-4.0 (e.g., 1.5-2.0) times the diameter of the pipe which can approximated by the diameter of the sealing discs of the PIG At this separation, two coupling paths exist between the transmitter and the receiver. A direct coupling path, where the amplitude of the field is significantly attenuated and become extinct at approximately two pipes diameter. Another, indirect coupling path where the magnetic field defuses into the walls of the pipe, propagates through it, then re-enter the pipe and measured by the receivers. In the remote field region, the indirect field is dominant.

Due to the need for low operating frequencies for electromagnetic fields to penetrate ferromagnetic pipe walls and the considerable distance between transmitter and receiver coils, the induced voltage on the receiver coils is significantly weakened. This results in a low signal strength, compromising the ability to detect and accurately characterize defects. By incorporating magnetic fluxgate sensors as the receivers 124, the LFEC sensor system 114 configured as a LF-RFEC sensor system can provide quantitative measurements with azimuthal information in contrast to typical RFEC tools can only provide qualitative measurements, estimating the average remaining conductive material in the measured region without azimuthal information.

Experimental tests were carried out to assess both a single receiver approach (FIG. 3A) and the array of smaller receivers approach (FIG. 3B). The tests used fluxgate magnetometers rather than sensor coils. System parameters varied during the study included the transmitter coil 128, the sensing elements 124, the operating frequency, and size of defects. Solenoid transmitter coils and fluxgate magnetometers DRV425 from Texas Instruments are used.

Several orientation designs were also considered for each of the sensing elements in both axial and radial directions with respect to the pipe's inner surface. In the axial direction, the sensing axis was parallel to the pipe's inner surface, while in the radial direction, the sensing axis was perpendicular to the pipe's inner wall. This allowed investigation of the effects of the orientation on the sensitivity and accuracy of each sensing element. By measuring the magnetic field components along both directions, the performance of the sensors was compared to evaluate their suitability.

In tests of single receiver approach (FIG. 3A), the 3D radial and axial phase distributions of magnetic measurements were studied and analyzed. It was observed that radial sensors exactly in the center leads to tiny little movements/vibration in the radial directions resulting in a noticeable phase change in the measurements, masking small defect features and impacting proposed localizing method. The test results indicated that the single receiver approach was ineffective in detecting small flat bottom hole defects machined on the outer surface of the pipe with various diameters and depth.

In tests of the array of smaller receivers approach (FIG. 3B), better results were observed. Use of miniature single-axis fluxgate magnetometers with relatively high measuring range (+/−2 mT) allowed the sensors to be placed near to the wall of the pipe without concern of magnetic saturation. The DRV425 from Texas Instruments offers a bandwidth measurement range of 47 kHz with built in accurate reference and internal compensation coil and conditioning circuit. The sensor is available with a body size of 4 mm×4 mm.

The LF-RFEC with an array of sensors demonstrated the potential to detect targeted small defects and pinholes. For example, outer pinholes as low as 4 mm in diameter with 3 mm depth (50%) and slots as small as 10 mm long with 1 mm width and 1.5 mm depth were detectable. Operation at low frequencies (e.g., <70 Hz) was necessary for proper detection of outer surfaces. In addition, the spacing between transmitter and the receivers needed to be more than at least 150% of the diameter of the pipe. Thus, the speed of the system is typically slow (0.1 m/s-0.4 m/s)

Both axial and radial configurations provided similar performance in terms of detection for the tested defects. Axial configuration is easier to work with as the received magnetic field strength in this orientation is an order of magnitude higher than the radial configuration, hence measurement will be more achievable with axial configuration. Radial configuration is more sensitive to defects, hence the amplitude of anomalies collected by the radial configuration are larger than the ones collected by the axial configuration however, radial measurement are noisier than axial measurements.

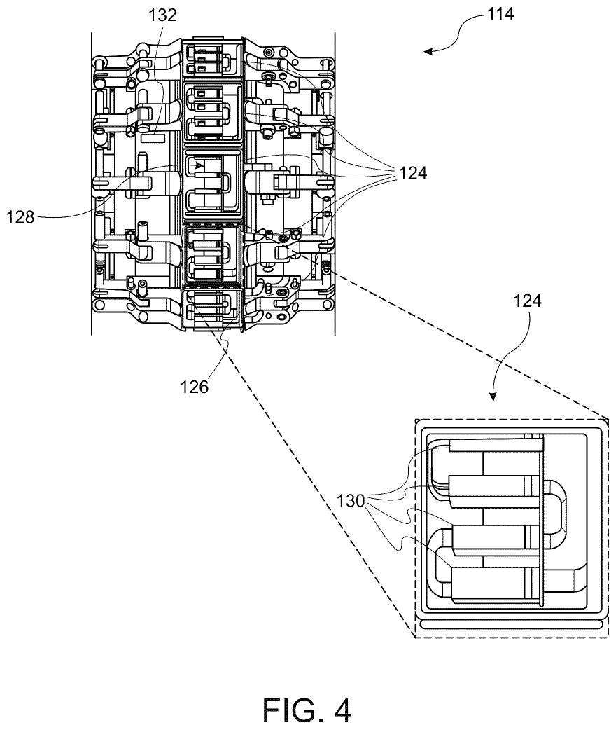

FIG. 4 is a schematic side view of a prototype LF-NFEC sensor system 114 of the eddy current testing system 100. As mentioned above, the LFEC sensor system 114 interacts with and covers the OD of a metal pipe for the detection of external wall losses. A relatively LF (e.g., 30-1000 Hz) is used in the measurement system in order to penetrate into the metal pipe. The LFEC sensor system 114 can use a transmitter coil and a receiver, or an array of receivers. As illustrated, the LFEC sensor system 114 includes multiple receivers 124, a LF routing board 126, and a transmitter coil 128. It also includes a control unit 132 and associated battery.

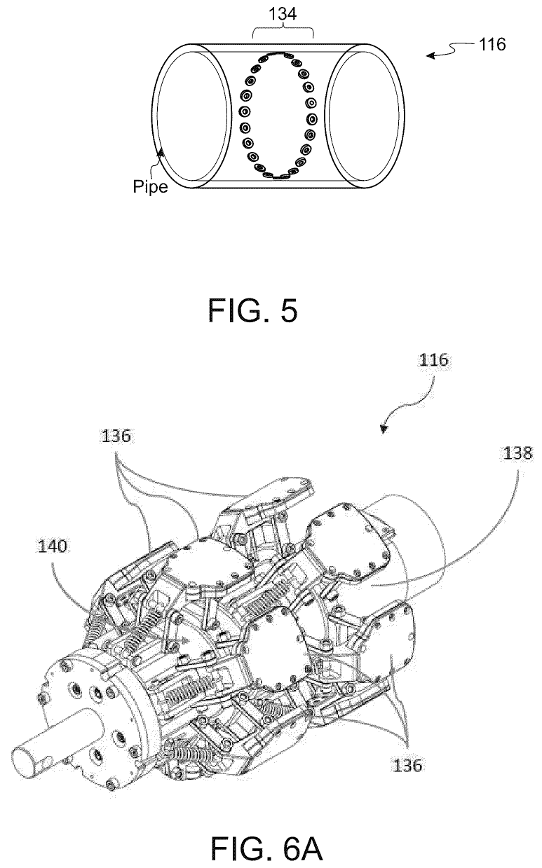

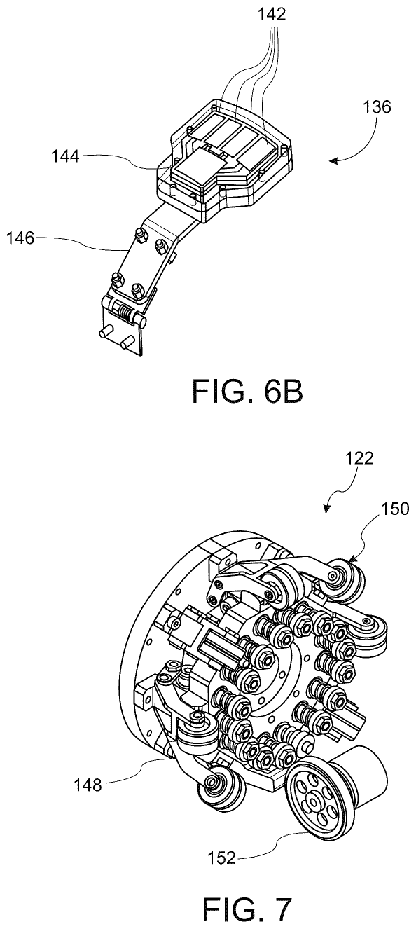

In the LFEC sensor system 114, the receivers 124 are sensor modules that incorporate fluxgate sensors. Some systems use receiver coils rather than fluxgate sensors. Each receiver 124 includes four fluxgate sensors 130 and an analogue to digital converter (ADC) employed to convert the analog sensor readings into digital. The prototype included total 48 analogue sensors and 12 ADC chips. Flexi-rigid PCB was used in order to optimize the placement of the PCB and the packaging of the electronics. The sensor modules included vibration sensors and were mounted on metallic arms that maintain consistent sensor positioning relative to the pipe surface. The metallic arms 146 help position the sensors accurately within the pipeline for effective inspection and are described in more detail with respect to FIGS. 6A and 6B.

The transmitter coil 128 is axially aligned with pipe. The receivers 124 are distributed circumferentially with a prescribed spacing between each two adjacent receivers 124 and at the same distancing from the center of the transmitter coil 128. This configuration and operation at the higher end of the LF-NFEC frequencies of 30-1000 Hz provides a system that is effective at higher speeds, which can accommodate for the native field conditions with high product flow rate.

The LF routing board 126 is used to distribute and organize high number of connections. The LF routing board 126 also employs a buffering circuit for the data lines to allow communications over relatively long cable used to connect to the control unit 132.

The transmitter coil 128 is mounted around the center of the PIG 112. In the prototype, litz wires were used in the winding of the transmitter coil 128 in order to reduce losses and improve the quality of data. The AC drive of the transmitter coil 128 comes from an inverter employed in the control unit 132.

The LFEC sensor system 114 includes control unit 132 and associated battery. The control unit 132 includes three printed circuit boards (PCB)—a processing board, a power supply board and a motherboard.

The processing board is responsible for data processing, data logging, speed and time measurement, and the overall system control. It includes an SD-card interface to provide additional data storage beyond the built-in memory of the processing board. The processing board also includes communication interfaces including USB, Ethernet, I2C, UART and SPI implemented to communicate with different parts of the system. The processing board also includes an encoder interface with a digital counter circuit designed to provide accurate and fast measurement of speed and distance based on signals from the encoder of the suspension wheels sensor system 122. The processing board also includes a real time clock employed for time keeping and powered by a coin-cell battery and an inertial measurement unit (IMU) integrated for measurements of pitch, roll and yaw angles.

The power supply board includes several DC-DC converters and power driver employed to generate the necessary conditioned system voltages used for internal and external loads as well as the power drive signal for the LF TX coil.

The motherboard is the base board that accommodates the processing board and the power supply board. It also contains connectors to the battery pack as well as data and power connections to the HF and LF electronics and the encoder. The motherboard provides some protection features such as reverse input (battery) voltage and over-current protection is applied to external voltages.

FIG. 5 is a schematic view of the sensor configuration for a HFEC sensor system 116. Multi-layer planar PCB sensor coils 134 are circumferentially distributed at equal radial distances from the center of the pipe to be inspected, for example, with 15° spacing. To achieve a reliable detectability possible, the sensor coils are located as close as possible to the inner surface of the pipe, but not directly touching the inner surface of the pipe. The adjustment of the size, and the number of coils as well as the number of the axial layers of the sensor coils, can provide sensitivity of full wall losses or defects that lie on the inner side of various size pipes. The use of PCB sensor coils 134 eliminates the need for excitation sources. This results in a simpler, more robust design capable of withstanding harsh environmental conditions, such as high temperatures or vibrations.

The HFEC sensor system 116 uses inductive sensing principles and inductance-to-digital converters (IDCs) to provide an inspection method without the use of an excitation source presented either by a transmitter coil or a magnet. Inductive sensing works on the principle of electromagnetic coupling between a sensor coil and the metal target to be detected and/or inspected, which in this case the inner wall of the pipe. When the metal target enters the electromagnetic field induced by the sensor coil, some of the electromagnetic energy is transferred into this target. This transferred energy leads to a circulating electrical current (eddy current). In return, the eddy current flowing in the metal target induces a reverse electromagnetic field on the sensor coil, which changes the sensor coil's effective inductance. This eddy current, and accordingly the change in the inductance of the sensor coil is a function of the distance, size, composition, and shape of the metal target, i.e. pipe. Depending on the defects' existence, number, type, size, and position, the inductance will have different values for each sensing coil. Monitoring and quantifying these changes allows for detecting and characterizing the defects. The elimination of an excitation source delivers various benefits including low cost, high reliability, low power consumption, and robust operation even in high-temperature situations.

The HFEC sensor system 116 employs EM fields with a relatively HF, up to 10 MHz (e.g., 10-100 KHz to 10-100 MHz) to enhance detection accuracy defects on pipe inner walls. These frequencies increase the magnitude of the eddy currents providing an improved signal-to-noise ratio and defect characterization performance compared to other operating frequency ranges.

FIG. 6A is a perspective view of a prototype HFEC sensor system 116 which can fully characterize the size, location (azimuthal angle), and level (thickness) of inner and full metal losses. Use of recently developed IDCs has enabled use inductive sensing for proximity sensing applications. As illustrated, the HFEC sensor system 116 includes HF sensor modules 136 and an HF routing board 138. It also includes a control unit 140 and associated battery.

FIG. 6B is a more detailed view of a single HF sensor module 136. Each HF sensor module 136 includes four sensing coils 142 are driven by one LDC driver 144. The prototype included 12 LDC drivers. The HF sensor module design used flexi-rigid PCB in order to allow controlled placement of sensing coils. The sensors are securely housed within a capsule employing non-metallic materials like resin or acrylic. The use of non-metallic materials such as resin or acrylic can contribute to the overall effectiveness of the sensor modules' sealing and protection while providing corrosion resistance, lightweight design, thermal insulation, and electrical insulation.

The resin housing is further enhanced with a sealing mechanism of a resin compound applied to envelop and protect the sensing coils. The resin compound can seal the sensors effectively and provide an additional layer of physical protection. The combination of the resin compound and the housing offers robust defense against potential hazards within the pipeline.

Low-friction materials like polyurethane or PTFE are used to make covers which are employed as an extra protective layer. These covers are fastened securely using screws over rubber O-rings. The rubber O-rings play a crucial role in creating a reliable seal. This dual-layer sealing approach provides redundancy, enhancing the PIG's ability to maintain a secure and impenetrable enclosure for the sensors.

Each HF sensor module 136 includes vibration sensors and is mounted on metallic arms 146 that maintain consistent sensor positioning relative to the pipe surface. The metallic arms 146 help position the sensors accurately within the pipeline for effective inspection. The metallic arms are equipped with springs 151 which help maintain consistent and fixed distances between the sensing sensors and the inner surface of the pipe. Maintaining these fixed distances helps achieve precise and uniform inspection results across the entire length of the pipeline. The springs are selected and calibrated to provide the necessary tension to keep the sensing capsules at an appropriate distance from the inner pipeline wall. This tension helps in maintaining a constant separation, even when the PIG encounters variations in the pipeline's diameter, roughness, or other irregularities. In essence, the springs act as a dynamic buffer, adapting to the nuances of the pipeline while preserving the desired proximity.

FIG. 7 is a perspective view of the suspension wheels sensor system 122 of the eddy current testing system 100 of FIGS. 1A and 1B. The suspension wheels sensor system 122 includes a network of arms 148 that serve as robust yet flexible connectors, offering stability and maneuverability simultaneously. Integrated within these arms are springs that enable the attachment to adapt to changes in pipeline diameter, surface roughness, and other subtle variations. They also serve to dampen shocks and vibrations, allowing for a smoother and more reliable inspection process.

Wheels 150 are mounted on the end of most of the arms 148 to provide reliable contact with the pipeline's inner surface. The wheels 150 help ease movement along the pipe and also assist in maintaining the HFEC sensor systems alignment. The wheels 150 come into direct contact with the pipeline, supporting the PIG's forward movement and ensuring that the inspection remains consistently close to the pipeline's interior.

An encoder 152 is mounted on one of the wheels 150. The encoder 152 includes a wheel that maintains continuous contact with the inner surface of the pipeline throughout the inspection process. The length of the encoder arm and characteristics of its spring are chosen to provide this continuous contact.

As previously discussed, systems can be assembled that combine different packages of low frequency and high frequency eddy current sensors.

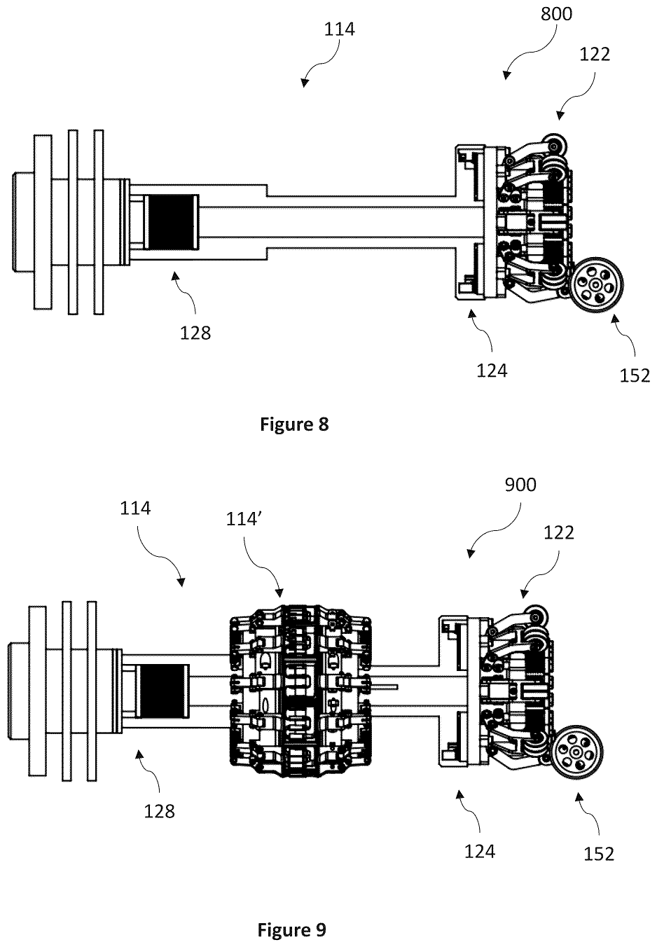

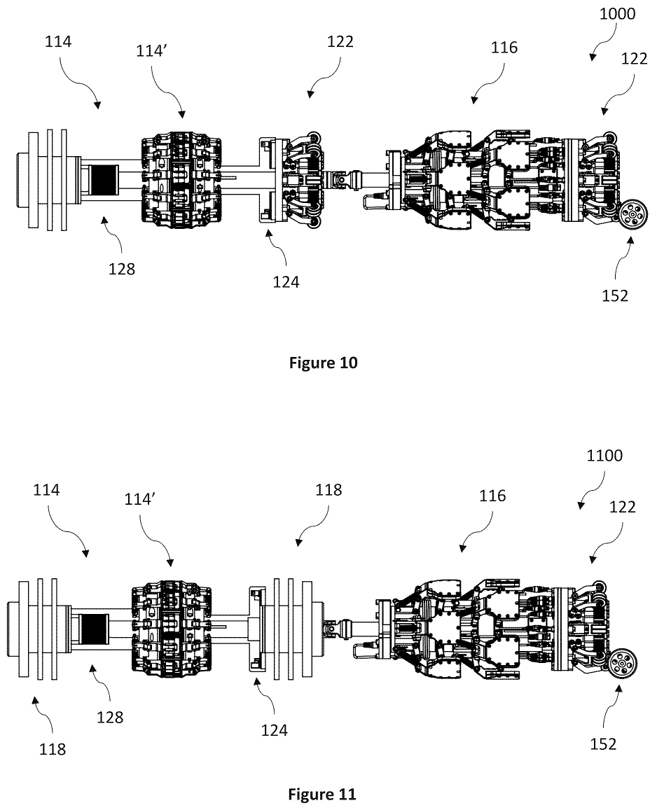

FIGS. 8-11 present several different systems based on eddy current sensors. FIG. 8 is a schematic of a system 800 with LF-RFEC sensors 114. The system 800 includes a receiver 124, a transmitter 128, a suspension wheel system 122, and an encoder 152. FIG. 9 is a schematic of a system 900 with LF-RFEC sensors 114 and LF-NFEC sensors 114′. The system 900 includes a receiver 124, a transmitter 128, a suspension wheel system 122, and an encoder 152. FIG. 10 is a schematic of a system 1000 with LF-RFEC sensors 114, LF-NFEC sensors 114′, and HFEC sensors 116. The system 1000 includes two suspension wheel systems 122, and an encoder 152. FIG. 11 is a schematic of a system 1100 with LF-RFEC sensors 114, LF-NFEC sensors 114′, and HFEC sensors 116. The system 1000 includes sealing discs 118 associated with the LFEC module. A suspension wheel system 122 and an encoder 152 are associated with the HFEC module.

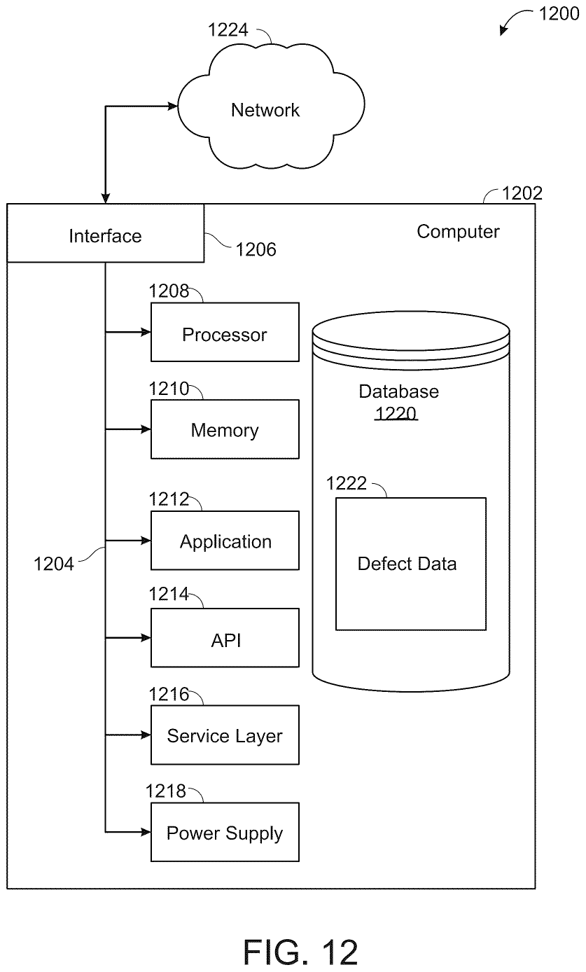

FIG. 12 is a block diagram of an example data processing system 1200 used to provide computational functionalities associated with described algorithms, methods, functions, processes, flows, and procedures described in the present disclosure. For example, the data processing system 1200 can be configured for controlling the sensor systems described with respect to FIGS. 1A-7. The data processing device 1202 can include input devices such as keypads, keyboards, and touch screens that can accept user information. Also, the data processing device 1202 can include output devices that can convey information associated with the operation of the data processing device 1202. The information can include digital data, visual data, audio information, or a combination of information. The information can be presented in a graphical user interface (UI) (or GUI).

The data processing device 1202 can serve in a role as a client, a network component, a server, a database, a persistency, or components of a computer system for performing the subject matter described in the present disclosure. The illustrated data processing device 1202 is communicably coupled with a network 1224. In some implementations, one or more components of the data processing device 1202 can be configured to operate within different environments, including cloud-computing-based environments, local environments, global environments, and combinations of environments.

The data processing device 1202 can receive requests over network 1224 from a client application (for example, executing on another data processing device 1202). The data processing device 1202 can respond to the received requests by processing the received requests using software applications. Requests can also be sent to the data processing device 1202 from internal users (for example, from a command console), external (or third) parties, automated applications, entities, individuals, systems, and computers.

Each of the components of the data processing device 1202 can communicate using a system bus 1204. In some implementations, any or all of the components of the data processing device 1202, including hardware or software components, can interface with each other or the interface 1206 (or a combination of both), over the system bus 1204. Interfaces can use an application programming interface (API) 1214, a service layer 1216, or a combination of the API 1214 and service layer 1216. The API 1214 can include specifications for routines, data structures, and object classes. The API 1214 can be either computer-language independent or dependent. The API 1214 can refer to a complete interface, a single function, or a set of APIs.

The service layer 1216 can provide software services to the data processing device 1202 and other components (whether illustrated or not) that are communicably coupled to the data processing device 1202. The functionality of the data processing device 1202 can be accessible for all service consumers using this service layer. Software services, such as those provided by the service layer 1216, can provide reusable, defined functionalities through a defined interface. For example, the interface can be software written in JAVA, C++, or a language providing data in extensible markup language (XML) format. While illustrated as an integrated component of the data processing device 1202, in alternative implementations, the API 1214 or the service layer 1216 can be stand-alone components in relation to other components of the data processing device 1202 and other components communicably coupled to the data processing device 1202. Moreover, any or all parts of the API 1214 or the service layer 1216 can be implemented as child or sub-sensor systems of another software sensor system, enterprise application, or hardware sensor system without departing from the scope of the present disclosure.

The data processing device 1202 includes an interface 1206. Although illustrated as a single interface 1206 in FIG. 12, two or more interfaces 1206 can be used according to implementations of the data processing device 1202 and the described functionality. The interface 1206 can be used by the data processing device 1202 for communicating with other systems that are connected to the network 1224 (whether illustrated or not) in a distributed environment. Generally, the interface 1206 can include, or be implemented using, logic encoded in software or hardware (or a combination of software and hardware) operable to communicate with the network 1224. More specifically, the interface 1206 can include software supporting one or more communication protocols associated with communications. As such, the network 1224 or the interface's hardware can be operable to communicate physical signals within and outside of the illustrated data processing device 1202.

The data processing device 1202 includes a processor 1208. Although illustrated as a single processor 1208 in FIG. 12, two or more processors 1208 can be used according to implementations of the data processing device 1202 and the described functionality. Generally, the processor 1208 can execute instructions and can manipulate data to perform the operations of the data processing device 1202, including operations using algorithms, methods, functions, processes, flows, and procedures as described in the present disclosure.

The data processing device 1202 also includes a database 1220 that can hold data for the data processing device 1202 and other components connected to the network 1224 (whether illustrated or not). For example, database 1220 can be in-memory or a database storing data consistent with the present disclosure. In some implementations, database 1220 can be a combination of two or more different database types (for example, hybrid in-memory and conventional databases) according to implementations of the data processing device 1202 and the described functionality. While database 1220 is illustrated as an internal component of the data processing device 1202, in alternative implementations, database 1220 can be external to the data processing device 1202.

The data processing device 1202 also includes a memory 1210 that can hold data for the data processing device 1202 or a combination of components connected to the network 1224 (whether illustrated or not). In some implementations, memory 1210 can be a combination of two or more different types of memory (for example, a combination of semiconductor and magnetic storage) according to implementations of the data processing device 1202 and the described functionality. While memory 1210 is illustrated as an internal component of the data processing device 1202, in alternative implementations, memory 1210 can be external to the data processing device 1202.

The application 1212 can be an algorithmic software engine providing functionality according to implementations of the data processing device 1202 and the described functionality. For example, application 1212 can serve as one or more components, sensor systems, or applications.

The data processing device 1202 can also include a power supply 1218. The power supply 1218 can include a rechargeable or non-rechargeable battery that can be configured to be either user- or non-user-replaceable.

There can be any number of computers 1202 associated with, or external to, a computer system including the data processing device 1202, with each data processing device 1202 communicating over network 824. Further, the terms “client,” “user,” and other appropriate terminology can be used interchangeably, as appropriate, without departing from the scope of the present disclosure. Moreover, the present disclosure contemplates that many users can use one data processing device 1202 and one user can use multiple computers 1202.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more sensor systems of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based).

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random-access memory (RAM), read only memory (ROM), phase change memory (PRAM), static random-access memory (SRAM), dynamic random-access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks.

A number of embodiments of the systems and methods have been described. Nevertheless, it will be understood that various modifications may be made without departing from the spirit and scope of this specification. Accordingly, other embodiments are within the scope of the following claims.

What is claimed is:

1. A tool for pipe inspection, the tool comprising:
a pipe inspection gauge (PIG) with two sets of sealing disc, a housing positioned between the sealing discs, and a body with an axis positioned within the housing;
a low frequency eddy current (LFEC) sensor system mounted within the housing of the PIG around an outer circumference of the body of the PIG;
a high frequency eddy current (HFEC) sensor system with an upstream end, a downstream end, and an axis extending between the upstream end and the downstream end, the HFEC sensor system having a flexible joint at the upstream end, the flexible joint attached to the PIG, and
a suspension wheels sensor system attached to the downstream end of the HFEC sensor system.

2. The tool of claim 1, wherein the HFEC sensor system comprises a plurality of HF sensor modules circumferentially distributed at equal radial distances from the axis of the HFEC sensor system.

3. The tool of claim 2, wherein each HF sensor module comprises four sensor coils driven by an driver.

4. The tool of claim 3, wherein the sensing coils are multi-layer planar printed circuit board (PCB) sensor coils.

5. The tool of claim 3, wherein the sensors are housed within a capsule fabricated from non-metallic materials.

6. The tool of claim 2, wherein each HF sensor module comprises vibration sensors.

7. The tool of claim 6, wherein each HF sensor module is mounted on a metallic arm equipped with springs which bias the associated HF sensor module towards a fixed distances between the sensing sensors and the inner surface of the pipe.

8. The tool of claim 1, wherein the HFEC sensor system further comprises an HF routing board, a control unit, and a battery.

9. The tool of claim 1, wherein HFEC sensor system comprises a source generating electromagnetic fields with a frequency between 1 and 10 KHz.

10. The tool of claim 1, wherein the suspension wheels sensor system comprises a plurality of arms with integrated springs, and a plurality of wheels, each wheel mounted on one of the plurality of arms.

11. The tool of claim 10, wherein the suspension wheels sensor system further comprises an encoder mounted on one of the plurality of arms.

12. A tool for pipe inspection, the tool comprising:
a low frequency eddy current (LFEC) sensor system mountable within a housing of a PIG around an outer circumference of a body of the PIG;
a high frequency eddy current (HFEC) sensor system with an upstream end, a downstream end, and an axis extending between the upstream end and the downstream end, the HFEC sensor system having a flexible joint at the upstream end, the flexible joint attachable to the PIG, and
a suspension wheels sensor system attachable to the downstream end of the HFEC sensor system.

13. The tool of claim 12, wherein the HFEC sensor system comprises a plurality of HF sensor modules circumferentially distributed at equal radial distances from the axis of the HFEC sensor system.

14. The tool of claim 13, further comprising multi-layer planar printed circuit board (PCB) sensor coils.

15. The tool of claim 13, wherein each HF sensor module comprises vibration sensors.

16. The tool of claim 13, wherein each HF sensor module is mounted on a metallic arm equipped with springs which bias the associated HF sensor module towards a fixed distances between the sensing sensors and the inner surface of the pipe.

17. The tool of claim 12, wherein HFEC sensor system comprises a source generating electromagnetic fields with a frequency between 1 and 10 KHz.

18. The tool of claim 12, wherein the suspension wheels sensor system comprises a plurality of arms with integrated springs, and a plurality of wheels, each wheel mounted on one of the plurality of arms.

19. The tool of claim 18, wherein the suspension wheels sensor system further comprises an encoder mounted on one of the plurality of arms.
